(19) United States
(12) Patent Application Publication
(10) Pub. No.: US 2018/0202072 A1
(43) Pub. Date: Jul. 19, 2018

(54) SEQUENCE-INDEPENDENT NUCLEIC ACID ASSEMBLY

(71) Applicant: The Regents of the University of California, Oakland, CA (US)

(72) Inventors: Todd Prentice Coleman, La Jolla, CA (US); Phillip Kyriakakis, La Jolla, CA (US)

(73) Assignee: The Regents of the University of California, Oakland, CA (US)

(21) Appl. No.: 15/746,371

(22) PCT Filed: Jul. 20, 2016
(86) PCT No.: PCT/US16/43104

§ 371 Date: Jan. 19, 2018

Related U.S. Application Data
(60) Provisional application No. 62/194,730, filed on Jul. 20, 2015.

(51) Int. Cl. C40B 50/14; C12Q 1/68; B01J 19/00

(52) U.S. Cl. CPC C40B 50/14; C12Q 1/68; B01J 2219/00605; B01J 2219/00722; B01J 2219/00729; B01J 19/0046

(57) ABSTRACT

Disclosed is a method for assembling nucleic acid fragments in a sequence-independent way. The method entails attaching a nucleic acid fragment to a solid phase, and contacting the first nucleic acid fragment with a second nucleic acid fragment such that the second nucleic acid fragment is ligated to the first nucleic acid fragment in a directional way. These steps can be repeated multiple times to assemble additional nucleic acid fragments. The method can further include a step of recovering the ligated nucleic acid to obtain the assembled nucleic acid.

### CROSS-REFERENCE TO RELATED APPLICATIONS

This patent document claims benefit of priority of U.S. Provisional Patent Application No. 62/194,730, entitled “SEQUENCE-INDEPENDENT OLIGONUCLEOTIDE ASSEMBLY” filed on Jul. 20, 2015. The entire content of the aforementioned patent application is incorporated by reference as part of the disclosure of this patent document.

### TECHNICAL FIELD

This patent document relates to systems, devices, and processes for assembling nucleic acid fragments on a solid phase.

### BACKGROUND

This patent document relates to assembling nucleic acid fragments, particularly fragments that are difficult to be assembled using conventional technology. In general, it is challenging to synthesize large nucleic acids in one step due to the error rate of incorporating each additional nucleotide. Therefore, large size nucleic acids are assembled from smaller fragments that can be sequence verified. It is difficult to assemble certain sequences, such as sequences containing small fragments that do not have overlapping sequences or sequences containing many repeats. The technology disclosed herein solves these problems by providing a method to assemble nucleic acids in an orientation-specific, sequence-independent way on a solid phase.

### SUMMARY

Examples of implementations of the disclosed technology can be used to provide systems, devices and techniques for assembling nucleic acids in a way that is independent of the sequence of the final assembled nucleic acid. Some exemplary nucleic acids include but are not limited to DNA, RNA, locked nucleic acid (LNA), unlocked nucleic acid (UNA), peptide nucleic acids (PNA), and/or other nucleic acids (containing natural and unnatural/synthetic bases) can be assembled according to the disclosed technology. Combinations of these exemplary nucleic acid fragments can also be assembled such that the assembled nucleic acid comprises one or more of the types of the examples of the nucleic acids listed above.

The disclosed technology can be used to assemble nucleic acids sequentially in a directional manner, enabling the assembly of nucleic acid sequences that include repetitive sequences and sequences with high A/T or G/C content. The disclosed technology assembles nucleic acids using an end-to-end ligation or a blunt ligation technique and does not rely on the final product to contain sequences that can efficiently hybridize to each other. For example, the nucleic acid fragments to be assembled do not contain any overlapping sequences and can be single-stranded or double-stranded. The disclosed technology for assembling nucleic acids can be used in various applications including a personal nucleic acid printer, consumable reagents for gene synthesis, and a large scale/multiplexed gene synthesis platform.

The disclosed technology assembles nucleic acids using Solid Phase Ligation Chain

Reaction for Sequence Independent Nucleic Acid Assembly. Unlike the conventional technology for nucleic acid assembly, which requires the presence of unique overlapping sequences, the disclosed technology conducts sequential end-to-end or blunt ligation of nucleic acids and therefore, can be used to assemble both non-repetitive sequences and repetitive sequences. Solid Phase Ligation Chain Reaction for Sequence Independent Nucleic Acid

Assembly may be used to assemble single stranded or double stranded nucleic acids. The method includes enabling a polymerase, such as a DNA polymerase, to use an immobilized nucleic acid fragment as a primer to turn the first nucleic acid fragment into a double stranded nucleic acid or a primer that binds or anneals anywhere in the assembling nucleic acid.

The method disclosed herein can be implemented in various ways to include one or more of the following features. For example, a first nucleic acid fragment to be assembled can be covalently linked to a solid phase or non-covalently linked through hydrogen bonds, ionic interactions, hydrophobic forces or any combination of these, to a solid phase. Alternatively, the first nucleic acid fragment to be assembled can be linked to the solid phase by attaching or annealing to an anchoring oligo. The anchoring oligo may be single stranded, double stranded, partially double stranded and/or a single strand folded into partially double stranded forms (e.g. hairpins). Thermostable ligases with high optimal temperatures that ligate together two single stranded nucleic acids, two double stranded nucleic acids or a single strand to a double strand nucleic acid can be used. In some embodiments, the ligase has a working temperature of about 65° C., above 65° C. or below 65° C., such that the secondary structure formation during the reaction of the single stranded nucleic acid is minimized. In some embodiments, ligation can proceed using non-enzymatic means, such as cyanogen bromide, N-Cyanoimidazole, or 1,2,3-triazole. The 3′ end of the nucleic acid is covalently or non-covalently linked to the solid phase while the 5′ end of the nucleic acid is exposed and can be phosphorylated such that the nucleic acid fragments are sequentially assembled in a directional manner. Alternatively, the 3′ end of the nucleic acid is blocked or capped such that the additional nucleic acid fragments to be assembled can be extended from 5′ end only. In some embodiments, the first nucleic acid fragment or the anchoring oligo is linked to the solid phase in the middle and can form a hairpin. Once assembled, the nucleic acid can be cleaved off or made double stranded and/or amplified directly from the solid phase. In some embodiments, the nucleic acid assembly can be performed on an array that allows parallelizing nucleic acid assembly. In some embodiments, second and additional nucleic acid fragments to be assembled are not immobilized on the solid phase. For example, the second and additional nucleic acid fragments can be present in a solution or any other carrier that can be brought into contact with the first nucleic acid fragment attached to the solid phase.

In one aspect, the disclosure relates to a method of assembling nucleic acid fragments. The method entails the steps of: (a) attaching a first nucleic acid fragment to a solid phase; (b) contacting the first nucleic acid fragment with a second nucleic acid fragment such that the second nucleic acid fragment is ligated to the first nucleic acid fragment. The method can further include an additional step, (c) recovering the ligated nucleic acid to obtain the assembled nucleic acid. In some embodiments, the assembled nucleic acid is recovered by amplification or by cleaving the assembled nucleic acid off from the solid phase. In some embodiments, step (b) is carried out in the presence of a ligase. In some embodiments, the nucleic acid fragments are single-stranded and are assembled by end-to-end ligation. In some embodiments, the nucleic acid fragments are double-stranded and are assembled by blunt ligation. In some embodiments, the method can further entail a polymerization step such that one or more single-stranded nucleic acid fragments are polymerized into double-stranded nucleic acid fragments. The polymerization step can be performed before, after, or during the ligation step. In some embodiments, the nucleic acid fragments to be assembled are non-overlapping.

In some embodiments, a scaffold such as a protein or other molecule that binds to a solid phase in a conditional manner (e.g. under the control of certain temperature, pH, salt etc.) can be used. In some embodiments, the scaffold is not solid, and it can exist in solution during some reactive steps of this method (e.g. ligation, phosphorylation, etc.). In some embodiments, the scaffold exists in solution during some steps and bound to a solid phase during other steps. For example, if a protein or nucleic acid serves as a scaffold, it may be soluble during a ligation step and then bound to the solid phase during a wash step. As one of ordinary skill in the art would understand, this can be achieved in several ways, such as thermoprecipitation, controlling the oxidation state of a nucleic acid bound to the solid phase by thiol groups, or other known methods that reversibly immobilize nucleic acids or modified nucleic acids onto a surface. Alternatively, nucleic acids may react (e.g. ligate, phosphorylate, etc.) in solution, followed by a step that makes the scaffold solid or reattaches the assembly to a solid phase prior to washing.

In some embodiments, after step (b) and before step (c), the method further comprises: removing excessive second nucleic acid fragment, e.g., by washing; phosphorylating the ligated nucleic acid; and contacting the ligated nucleic acid with a third nucleic acid fragment such that the third nucleic acid fragment is ligated to the second nucleic acid fragment. As one of ordinary skill in the art would understand, these steps can be repeated multiple times to assemble one or more additional nucleic acid fragments. In some embodiments, the ligation is carried out in the presence of a ligase.

In another aspect, disclosed herein is a system for assembling nucleic acid fragments. The system comprises a solid phase, a first nucleic acid fragment immobilized on the surface of the solid phase directly or indirectly via an anchoring oligo, and one or more nucleic acid fragments to be assembled. The system can further comprise a ligase and a kinase, which are brought into contact with the nucleic acid fragments at appropriate stages and removed when not needed. For example, the ligase is brought in during the ligation stage and removed by washing when the ligation is completed; and the kinase is brought in during the phosphorylation stage and removed by washing when the phosphorylation is completed. Additionally, the system can further comprise a polymerase for amplification or polymerization, and/or a nuclease for cleaving the assembled nucleic acid. Ligations also can occur non-enzymatically. The solid phase can have a flat surface, a circular surface or a sphere surface.

In another aspect, a nucleic acid or gene printer is configured to perform operations of the disclosed methods. The nucleic acid printer can be configured to print ready-to-use plasmids, chromosomes, or nucleic acid materials. The nucleic acid printer may use microfluidics and/or array technology to facilitate assembly.

In another aspect, an inkjet nucleic acid printer is configured to perform operations of the disclosed methods, to spot nucleic acids and reagents for assembly of nucleic acids on a plate. The inkjet nucleic acid printer can be implemented in various ways to include one or more of the following features. For example, the inkjet nucleic acid printer can be configured to enable nucleic acid recovery through aspiration. The inkjet nucleic acid printer can be configured to receive a desired nucleic acid sequence; and print the received nucleic acid sequence as linear nucleic acids, as double-stranded nucleic acids, or be taken from the solid phase in a way that forms more complex structures (e.g., DNA/RNA origami, Triplex-Helix, G-quadruplex, or parallel stranded nucleic acids). The inkjet nucleic acid printer can be configured to ligate the printed linear nucleic acid fragments into circular nucleic acids, such as double-stranded plasmid DNA or a circular single-stranded nucleic acid.

In another aspect, a semiconductor, glass or other “chip” or “array” device is configured to perform operations of the disclosed method. It is within the purview of one of ordinary skill in the art to use any suitable device to carry out the application of assembling nucleic acids. The suitable device can take any size or shape, and has a scaffold or solid surface to attach or to link the nucleic acids to be assembled. In some embodiments, a scaffold is a protein or other molecule that binds to a solid phase in a conditional manner (e.g. under the control of certain temperature, pH, salt etc.). In some embodiments, the solid surface is a flat surface. In other embodiments, the solid surface is a circular or sphere surface. In some embodiments, the scaffold is not solid, and it can exist in solution during some reactive steps of this method (e.g. ligation, phosphorylation, etc.). In some embodiments, the scaffold exists in solution during some steps and bound to a solid phase during other steps. For example, if a protein or nucleic acid serves as a scaffold, it may be soluble during a ligation step and then bound to the solid phase during a wash step. As one of ordinary skill in the art would understand, this can be achieved in several ways, such as thermoprecipitation, controlling the oxidation state of a nucleic acid bound to the solid phase by thiol groups, or other known methods that reversibly immobilize nucleic acids or modified nucleic acids onto a surface. Alternatively, nucleic acids may react (e.g. ligate, phosphorylate, etc.) in solution, followed by a step that makes the scaffold solid or reattaches the assembly to a solid phase prior to washing. In some embodiments, the device is a chip device, such as a semiconductor chip device, a bead, glass, a porous material, or a combination thereof. The chip device can be implemented in various ways to include one or more of the following features. For example, the chip device can include one area or one address on the chip that contains fully assembled nucleic acids; and other areas or addresses on the chip contain not fully assembled nucleic acids. The chip device can include microarrays with immobilized nucleic acids. The chip device can include sequencing flow cells or microfluidics. The chip device can include microarrays having nucleic acid fragments annealed to specific locations on the microarrays. Assembly at specific locations can be controlled through application of an electric field, current, or optical control at specific locations on the chip, which techniques are known in the art. An example includes applying an electric field on a specific address on a chip to control ion availability in solution, effectively turning reactions on and off electrically in a manner that a digital camera addresses one pixel at a time. These ions can include, but are not limited to hydrogen/hydroxyl ions, calcium, magnesium, or any ion that regulates the activity of an assembly factor. Assembly at specific locations can also be controlled by spotting reagents at specific locations, such as with inkjet printing. The chip device can be configured to generate small or large libraries of nucleic acid sequence variants.

In other aspects, methods, systems, and devices can be implemented for assembling nucleic acids using end-to-end ligation or blunt ligation according to the disclosed technology.

### BRIEF DESCRIPTION OF THE DRAWINGS

FIG. 1 illustrates a process for assembling nucleic acids according to solid-phase ligation chain reaction.

FIG. 2 illustrates that end to end ligation can be carried out by extending from a nucleic acid fragment directly attached to the solid phase (A) or extending from a nucleic acid fragment indirectly attached to the solid phase via an anchoring oligo immobilized on the solid phase (B).

FIG. 3A illustrates a process for making a double-stranded nucleic acid from an assembled single-stranded nucleic acid. FIG. 3B illustrates a process for end to end ligation of double-stranded nucleic acids. FIG. 3C illustrates a process for annealing double-stranded nucleic acid onto an anchoring oligo and extending the double-stranded nucleic acid using blunt ligation. FIG. 3D illustrates a process similar to FIG. 3C except for extending with single-stranded nucleic acid using end to end ligation.

FIG. 4 demonstrates array to array nucleic acid printing using solid-phase ligation chain reaction, using DNA as an example. Array to array printing can be done by spot printing (inkjet printing etc.) or by sandwiching the DNA to be assembled between the arrays. DNA can be synthesized on the array or spotted and attached onto an array (400). Sandwiching can take place once one set of DNAs are cleaved off of the array. The array containing cleaved oligos (401) to be assembled in buffer without enzyme are combined (by sandwiching or microfluidics etc.) with a “master plate” (402) containing DNA that is linked to the solid phase along with buffer+enzyme. Once combined, the DNAs will be assembled by ligation. Assembled DNA can be washed and cleaved off or the “master plate” can be reused to assemble additional DNA (403).

FIG. 5 illustrates different configurations of the first nucleic acid fragment or the anchoring oligo attached to the surface of the solid phase. FIG. 5A shows that the first nucleic acid fragment is attached to the surface of the solid phase at the 3′ end, blocking extension from the 3′ end. FIG. 5B shows that the first nucleic acid fragment is attached to the surface of the solid phase in the middle of the fragment and the 3′ end is blocked or capped such that additional nucleic acid fragments can be added to the 5′ end only. FIG. 5C shows that the first nucleic acid fragment is attached to the surface of the solid phase in the middle of the fragment and the fragment forms a hairpin.

### DETAILED DESCRIPTION

Synthesizing large nucleic acids can be accomplished by assembling smaller nucleic acid fragments that contain overlapping sequence. For example, overlapping sequences can be annealed and each nucleic acid to be assembled can be used as a primer to polymerize the overlapping nucleic acids, or can be polymerized by ligation, fusing them together by PCR or Gibson assembly etc. This conventional assembly method relies on the ability to generate unique overlapping sequences and sequences that are favorable to annealing at a specific condition (e.g. temperature, pH, salt concentration etc.). In addition, when assembling more than two fragments, several unique overlapping sequences that anneal under the same conditions (temperature, pH, salt concentration etc.) need to be found. Assembling nucleic acid fragments using unique overlapping sequences may not work well in assembling repetitive sequences due to the requirement of unique overlapping sequences. Also, the assembly reaction is done in solution, limiting its scalability. Moreover, when assembling nucleic acid fragments using unique overlapping sequences the user cannot always accurately predict how the nucleic acid fragments being assembled will anneal, especially when assembling large numbers of nucleic acid fragments. This can lead to failure in assembly reactions, which requires a redesign and synthesis of the nucleic acid fragments and their overlaps. Some sequences may not even be amiable to this process and require assembly through cloning several small fragments into a plasmid or genome one at a time. These constraints can lead to an approach to assemble by assembling several partial sequences, followed by a second, third (or more) cloning steps, usually after an initial attempt has failed. This can be very time consuming, requiring up to several weeks to obtain a desired sequence.

The technology disclosed herein largely avoids the limitations of the process of assembling nucleic acid fragments that require overlapping sequences. First, the disclosed technology is sequence-independent and does not depend on the nucleic acid fragments being assembled to have any overlapping sequence. Thus, the disclosed technology can be used to assemble repetitive sequences, or sequences with either unusually high or low G/C or A/T content. Moreover, because the assembly chemistry is “solid phase” the disclosed technology can be achieved “on chip” with minor modifications of off the shelf technologies. Thus, the disclosed technology is highly scalable to assembling thousands or millions of nucleic acid fragments, such as polynucleotides and oligonucleotides, in parallel. As used herein, “solid phase” means a material that is in a solid phase and the material has a surface such that a nucleic acid fragment can be attached directly or indirectly to the surface of the solid phase material.

The ability to assemble nucleic acid fragments in a sequence-independent manner has important implications on synthetic biology, a growing field in academia and industry. Synthetic and systems biology approaches often require systematically changing sequences in order to optimize or study some biological system. It is common that these changes are repetitive patterns of nucleic acid sequence, for example, testing the effects on copy number of nucleic acid binding sites on transcription or copies on amino acid sequences on binding strength. Yet, when current art is tested for failure rate, they are tested against random sequences or variants of a particular gene that does not contain repetitive elements. In addition, the synthetic biology field aims at bottom up approaches to making synthetic organisms/genomes, yet 51-78% of the mammalian genome consists of “repetitive sequences” (this does not even include the aforementioned repetitive patterns that can influence gene promoter strength or copy number etc.). While current technologies promise to deliver synthesis and assembly of thousands of unique nucleic acid sequences, they are fundamentally limited in their ability to assemble nucleic acids that are commonly desired in biology and synthetic biology. There is no existing technology known in the art that can assemble multiple nucleic acid fragments in an orientation specific manner that does not rely on overlapping nucleic acid sequences.

In addition to the need for assembling repetitive nucleic acid sequences for synthetic/molecular biology, there is a fledgling field utilizing nucleic acids as programmable material (for example, to make programmable nanoparticles for imaging or drug delivery). This field relies on the assumption that the nucleic acids being modeled or programmed can be synthesized/assembled. These nucleic acids based materials are made from patterns of sequences that are mostly repetitive and it is very likely that, even on a small scale, current technologies will not be able to assemble them. Moreover, when considering nucleic acid as a material, the correct sequence does not just need to be cloned once, it will need to be assembled on a scale of grams or even kilograms. In short, current technologies may not be able to be easily scaled up. The technology disclosed here allows scaling by changing the amount of solid phase and nucleic acid.

The disclosed technology works by assembling nucleic acids such as nucleic acid fragments through end-to-end ligation or blunt ligation, eliminating the need for overlapping fragments. This eliminates the dependence of the sequence of the two nucleic acid fragments as a factor for assembly. The technology disclosed herein achieves sequence-independent assembly once the nucleic acids such as nucleic acid fragments are immobilized on a solid phase. The disclosed technology includes several variations that can provide various advantages for specific applications.

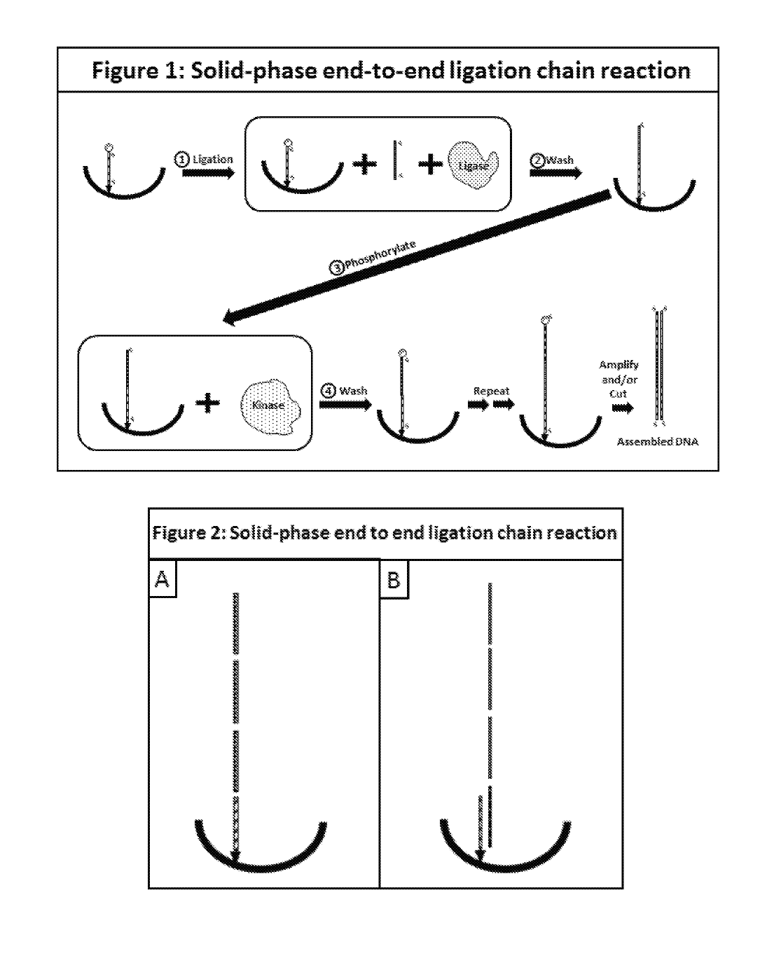

The disclosed technology can assemble nucleic acids through end-to-end ligation of single-stranded nucleic acids or blunt end ligation of double stranded nucleic acids by using nucleic acids covalently or non-covalently linked to a solid phase or scaffold. This is achieved by using a thermostable ligases with high optimal temperatures that ligate together single-stranded or double stranded nucleic acids. While other ligases can be used, several commercially available ligases work efficiently at or above 65° C. (such as Circligase™ ssDNA ligase) minimizing secondary structure of the single-stranded nucleic acids. Assembly is orientation specific due to the nature of nucleic acids having opposite polarity at 5′ end and 3′ end. A single-stranded nucleic acid fragment is linked to the solid phase on the 3′ end and contains a phosphate on the other end, the 5′ end (or it can be phosphorylated). Thus, the nucleic acid piece to be assembled (which is not phosphorylated) is only able to make a covalent bond with the nucleic acid that is attached to the solid phase in an orientation specific manner (5′ to 3′ linkage) (FIG. 1: STEP 1—Ligation). Excess nucleic acids and reagents can then be washed off the solid phase (FIG. 1: STEP 2—Wash), exposed ends can be phosphorylated (FIG. 1: STEP 3) and following another wash (FIG. 1: STEP 4) the process can be repeated again. Either single-stranded or double-stranded DNA can be assembled in this way. Orientation is specific with single-stranded DNA by blocking the 3′ end. This is achieved by attaching the DNA to the solid phase by the 3′ end. However, double-stranded DNA can be prepared by eliminating phosphates on one end specifically (for example, by cutting one end and dephosphorylating followed by cutting the other end) or by chemically blocking one of the 5′ ends. Alternatively, nucleic acids can be cleaved off, or made double stranded, and/or amplified from the solid phase. SDA may also be obtained by designing the last fragment to form a hairpin primer for SDA.

Nucleic Acid Fragment or Anchoring Oligo Immobilized to the Solid Phase

The method disclosed herein includes the use of an anchoring oligo or a first nucleic acid fragment that is bound to the surface of a solid phase through covalent coupling using carboxylic acids, primary naliphatic amines, aromatic amines, choromethyls (vinyl benzyl chloride), amides, hydrazides, aldehydes, hydroxyls, thiols, epoxys, disulfides groups, carbonyl amides, thioureas, sulfonamides, carboxamides, 1,2,3-triazole, or other linkage chemistries. In some embodiments, the anchoring oligo or the first nucleic acid fragment is bound to the solid phase via photoactivatable or photocleavable linkages, such as O-nitrobenzyl based linkers. For example, photocleavable linkages that selectively cleave the anchoring oligo or the first nucleic acid fragment can be used. In some embodiments, the anchoring oligo or the nucleic acid fragments to be assembled can have certain modifications, such as phosphates, fluorophores, fluorescent quenchers, spacers, phosphoorthate bonds, dideoxynucleic acids, biotin, or cap analogs (5′-5′ Dinucleo side Triphosphates).

The anchoring oligos may have various sequences that are complementary to the sequences of the nucleic acid fragments to be assembled. In some embodiments, the anchoring oligos have sequences that are completely complementary to the nucleic acid fragments to be assembled. In other embodiments, the anchoring oligos have sequences that are partially complementary to the nucleic acid fragments to be assembled. For example, the sequence of an anchoring oligo can have a mismatch to the sequence of the matching or corresponding nucleic acid fragment to be assembled, as long as the nucleic acid fragment to be assembled can still bind to the anchoring oligo and be immobilized on the solid phase. It is within the purview of one of ordinary skill in the art to select suitable sequences for the anchoring oligos.

### Annealing and Ligating

One advantage of the ligation at a higher temperature is to minimize possible inhibition or bias of the assembly due to secondary structure formation. Additionally, this method does not entail many steps and therefore is cost-efficient. If the extending nucleic acid strand is directly attached to the solid phase (FIG. 2A), a temperature that usually melts double-stranded nucleic acids can be used for washing and ligation, and no annealing step is required. Moreover, if the ligation is performed at a higher temperature and does not use any nucleic acid hybridization technique, the method does not require a stringent prescreening process to make sure that the nucleic acids to be assembled are not incorrectly hybridized to the solid phase.

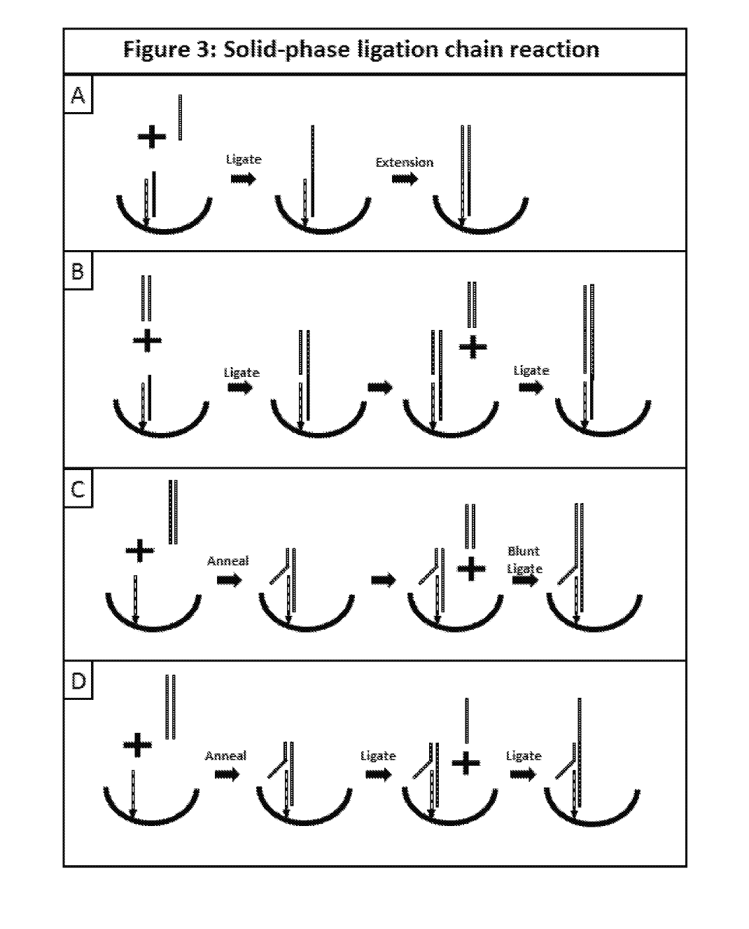

It is also possible with this invention to polymerize the nucleic acid into double stranded nucleic acid prior to the next round of end-to-end ligation of the incoming single strand. This may further block any base pairing of assembled nucleic acid. The assembled single-stranded nucleic acid can be made double-stranded during or after the assembly process. For example, the single stranded assembly may be carried out on a strand that is attached to the solid phase by hybridization to a solid phase oligo that is a reverse compliment or partial reverse compliment (FIG. 2B). This would enable a polymerase to extend from the solid phase nucleic acid making the assembled nucleic acid double stranded (FIG. 3A). Alternatively, the product may be made double stranded by annealing a primer to the single stranded nucleic acid at any point along the extending nucleic acid strand or to the anchoring oligo. When double-stranded nucleic acids are blunt end ligated, they do not have the same tendency to form interfering secondary structure at lower temperatures (FIG. 3B and 3C). In another embodiment, a double-stranded nucleic acid can be hybridized, serving as a substrate for single-stranded nucleic acids to assemble end-to-end (FIG. 3D). In this embodiment, the second strand of the nucleic acid can serve as a primer to make the product double-stranded.

Using lower temperatures and enzymes that function at lower temperatures is also possible. There are several ways to control the secondary structure formation during the assembly reaction. In some embodiments, secondary structure can be minimized by including single-stranded binding protein to the reactions. In some embodiments, nucleotides or nucleosides may be included in the reaction to minimize secondary structure or to adjust the annealing or self-annealing conditions. For example, if the nucleic acid to be assembled is high in A and/or T, A and/or T nucleotides or nucleosides can be added to modulate melting/annealing temperatures. Similarly, if the nucleic acid to be assembled is high in G and/or C, G and/or C nucleotides or nucleosides can be added to modulate melting/annealing temperatures. In some embodiments, ion concentrations can be adjusted (sodium, potassium, acetate, arginine, phosphate etc.), crowing agents such as polyethylene glycol (PEG) can be added, or other molecules that change the osmolarity can be added (such as glucose, sucrose, glycerol, BSA etc.), to modulate secondary structure formation.

### Recovery of Assembled Nucleic Acid

Once the nucleic acid is fully assembled there are several possible ways to recover the assembled nucleic acid. One way would be to simply cleave it off with a nuclease, leaving fully assembled nucleic acid. Alternatively, the nucleic acid can be amplified directly from the solid phase using single loop-mediated isothermal amplification (LAMP). A nucleic acid to be assembled may be designed to have a hairpin or loop on one end to facilitate amplification. Alternatively, strand displacement amplification (SDA) can be used for amplification. Primers for SDA can be designed to anneal the anchoring oligo or anywhere in the assembled nucleic acid. The 5′ end of the anchoring oligo may be protected to prevent polymerization or ligation at this site. SDA may result in a larger yield due to amplification, but would lead to single-stranded nucleic acid. Moreover, single-stranded nucleic acids can be melted off by heating. In addition, assembled nucleic acids can be amplified by PCR (DNA), transcription (DNA/RNA) or reverse transcription (RNA/DNA). If a fully assembled and ready to use nucleic acid is desired, it may be possible to assemble onto a plasmid backbone at any step during assembly. The plasmid can be ligated end-to-end or blunt ended to the extending nucleic acid chain. Alternatively, the plasmid can be bound to the solid phase and the nucleic acid fragments can be assembled onto the plasmid by end-to-end ligation or blunt end ligation. The plasmid may be ligated during the last step. This can be accomplished by cutting the plasmid with a restriction enzyme that leaves an overhang which can anneal to the solid phase oligo or by chewing back a few bases to expose single-stranded DNA that can anneal to the solid phase nucleic acids. Alternatively, the double-stranded plasmid can be melted, allowing it anneal to the assembled solid phase nucleic acid. The plasmid can be assembled in an orientation specific manner by selective phosphorylation of one end. Cleavage can be enzymatic, chemical, mechanical, thermal or using microwaves, radiowaves, x rays, UV, visible or IR light.

Each recovery method has specific advantages. For example, if one desires to recover DNA and immediately use for cloning into a plasmid (as in a “personal gene printer”), the reaction can be easily scaled to yield larger amounts of DNA (with a larger solid phase). The resulting double-stranded DNA can be digested with a restriction enzyme or used in one of many types of ligation independent or other cloning methods. This printer could in principle print ready-to-use plasmids. For example, a plasmid backbone can be designed so that single-stranded DNA can be amplified from a plasmid using SDA, making it linearized allowing for end to end extension onto the solid phase. Thus, the printed DNA can be ligated to the backbone and made double stranded with DNA polymerase, which can even be circularized by ligation after release from the solid phase. If the plasmid is double stranded, one end may be blocked to achieve assembly on a specific end of the plasmid. This DNA is ready for transformation which only requires as little as about 1 pg-100 ng of plasmid DNA. However it may be scaled up to produce micrograms, milligrams, grams or kilograms of nucleic acids.

When the application is to generate thousands of different nucleic acid fragments “on-chip”, the SDA recovery may be more optimal. This allows the reaction to be scaled down significantly since the recovery process is also an amplification step. It may even be further advantageous since the deprotection process may in theory be controlled by light. In this set up the light can selectively deprotect different areas of the chip thereby selectively recovering the assembled nucleic acid. One application for this approach can be that one area or one “address” on the chip may contain fully assembled nucleic acid, while the others may require more rounds of assembly. There may be other reasons to utilize this approach.

In some embodiments, recovery of the assembled nucleic acid may be controlled by light or other methods that control reactions in specific locations on the chip. One application for this could be that one area or one “address” on the chip may contain fully assembled nucleic acids, while the others may require more rounds of assembly. In this set up, the light could selectively deprotect different areas of the chip selectively recovering the nucleic acid. Another example would be applying an electric field on a specific address on a chip to control ion availability in solution, effectively turning reactions on and off electrically in a similar manner that a digital camera addresses one pixel at a time (CMOS technology). These ions can include, but are not limited to hydrogen/hydroxyl ions, Calcium, Magnesium, or any ion that regulates the activity of an assembly factor.

As one of ordinary skill in the art can appreciate, the assembled nucleic acid can be recovered by various means. For example, the method disclosed herein can include recovery of the nucleic acid using mechanically, for example using sonication. The method can include recovery of the nucleic acid thermally by heating or optical absorption/excitation. The method can include recovery of the nucleic acid chemically by enzyme cutting or by using redox chemistry or other chemical methods. The method can include recovery of the nucleic acid biochemically, such as through biotin elution. The method can include recovery of the nucleic acid by using photolabile or thermolabile groups.

It should be appreciated that the nucleic acid to be assembled can be one type of nucleic acid, while the nucleic acid that is amplified or copied from the solid phase may be a different type of nucleic acid. For example, an assembled DNA can be reverse transcribed into an RNA. In addition, multiple rounds of amplification from the solid phase may be utilized.

### Exemplary Applications of the Disclosed Technology

The design according to the disclosed technology would work well in a format for single gene assembly, making a nucleic acid/gene printer when combined with an oligosynthesizer. The disclosed technology could serve as a standalone device in a lab that prints desired genes.

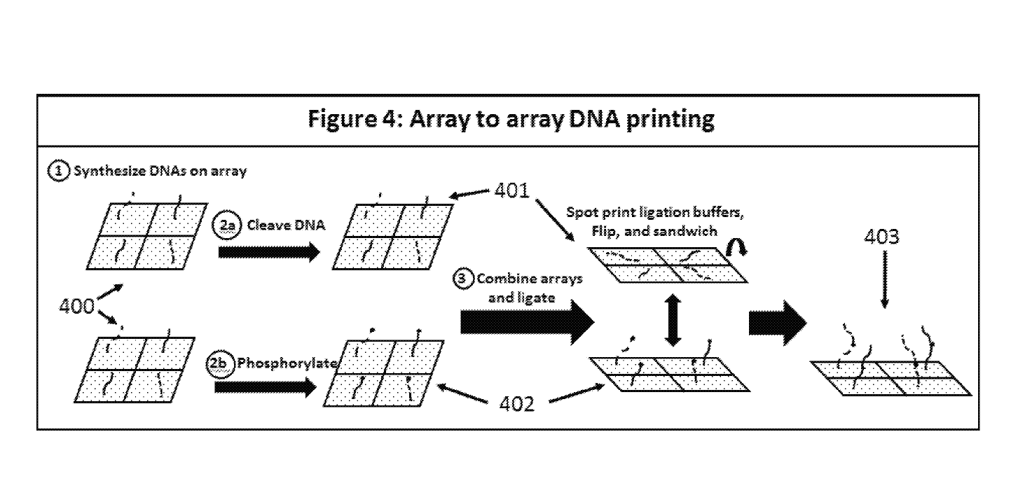

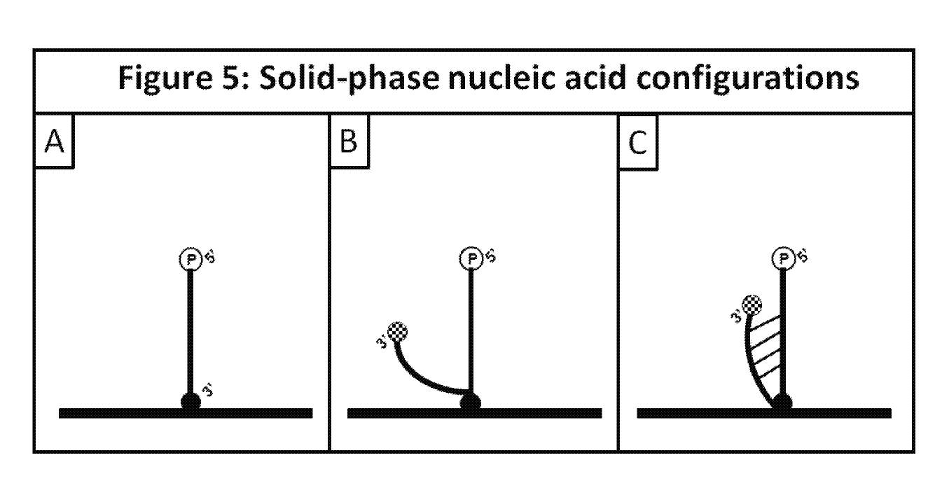

The disclosed technology also allows for parallelizing nucleic acid assembly as shown in FIG. 4. It is possible to spot reagents for assembly of nucleic acid on the plate (using inkjet printing technology for example) instead of a purely microfluidics approach. Nucleic acids can then be recovered through aspiration. For example, the same process as shown in FIG. 1 can be applied on an array. First nucleic acids can be synthesized on an array or in general the nucleic acid for assembly can be linked to the array (FIG. 4: 400). Then, using a “master array plate” (bottom of FIG. 4, 402 and 403) where nucleic acid is assembled, plates containing the next nucleic acid for assembly can be cleaved (FIG. 1: STEP 2, FIG. 4: STEP 2a) generating an array with nucleotides that are now unlinked from the solid phase (401). These unlinked arrays are then sandwiched onto the master array plate with enzymes and reagents necessary for ligation. Alternatively, the nucleic acid to be assembled with enzymes and reagents necessary for ligation can be spot printed, reacted and washed as in FIG. 1. To increase the efficiency of ligation and correct assembly, nucleic acid for assembly can be amplified from the array using SDA or PCR, generating a large excess of single or double stranded nucleic acids (excess compared to the master plate). For this approach, resulting nucleic acid for assembly would be phosphorylated, but can easily be dephosphorylated with a phosphatase prior to assembly onto the next solid phase. The phosphatase can be removed prior to contact with the nucleic acids attached to the solid phase by heat, affinity purification or other means to prevent dephosphorylation on steps where assembly would be inhibited by phosphatase activity. These same approaches can be applied to assembly of other nucleic acids, such as RNA, LNA, UNA, PNA etc. or other natural or unnatural bases, such as methyl-cytosine, and unnatural/synthetic bases. These bases can base pair normally or may base pair unnaturally, such as with d5SICS and dNaM.

According to the technology disclosed herein, a “personal” lab nucleic acid printer could print ready to use plasmids. For example, a plasmid backbone can be designed so that only single strand contains a phosphate allowing for control of the orientation of the assembled nucleic acid is ligated to the plasmid. Alternatively, if a fixed orientation is not necessary, nucleic acids and plasmids to be assembled can contain phosphates on both ends during ligation steps.

The single-stranded nucleic acid assembled according to this invention can be made double stranded through a number of methods. These can include, but are not limited to PCR or annealing to its reverse compliment. Thus, the printed nucleic acid would be ligated to the backbone and made double stranded, which can easily be circularized by ligation after release from the solid phase. This nucleic acid would be ready for transformation which only requires as little as 1 pg-100 ng of plasmid nucleic acid. If the application was to generate thousands of different nucleic acids “on-chip”, the SDA or PCR recovery may be most optimal. This would allow the reaction to be scaled down significantly since the recovery step was also an amplification step.

The disclosed technology, exemplified by different ways of nucleic acid assembly detailed above, has many applications for synthesizing nucleic acids for sale to academia as well as industry. There are three broad applications. One is to use this technology as part of a “personal gene synthesis machine”, that would allow a user to enter a desired nucleic acid sequence and have it “printed”. The entire plasmid can be designed from scratch, custom fit for the gene or application of interest, and can be “printed” as linear nucleic acid and even ligated by the gene printer into circular plasmid nucleic acid. An end user or a robot can then transform a custom plasmid; no other cloning steps would be necessary. Alternatively, it can combine the assembled nucleic acid with an existing plasmid, a piece of a plasmid, genomic DNA, mRNA, lcRNA, siRNA and/or other nucleic acid sources.

A second application includes using the same concept, scaled up with an “on-chip” design. This would enable the generation of thousands of long nucleic acid sequences or genes in parallel. This design could be used in a device that is favored by gene synthesis industry due to the high volume of production. This may be combined with an “on-chip” synthesis of nucleic acids—in a single device. There is a need for generating large libraries of nucleic acid sequence variants for understanding some biological phenomena or to optimize some biological process (e.g. optimizing an enzyme to improve bio-fuel production). The disclosed technology satisfies this need.

A third application is using nucleic acids as a material, such as in nanoparticles, for medical/drug delivery, bio/chem-sensors etc. Nucleic acids are easy to chemically/computationally design and model, allowing nucleic acid sequences to be programmed to perform a vast array of functions. For these applications, nucleic acids must be assembled in a way that is not restricted by what sequences can be assembled. Unlike genes that can code for the same protein with an altered sequence (due to redundant codon usage), when working with nucleic acids as a chemical/polymer (versus as gene), it is required to have the exact sequence as the computer model for testing a hypothesis or to have the exact chemical/material properties as the computer model. It is desirable to have an inexpensive way to synthesize large amounts of nucleic acid. Having a scalable system that can produce any sequence of interest is highly desirable and the disclosed technology satisfies these requirements.

A fourth application is for the synthesis of nucleic acids for information or data storage. Nucleic acids can last millions of years and the ability to read them will not become obsolete as do human made technologies. Therefore, there is growing interest in using nucleic acids as a way to store information. However, in order to encode digital information it is highly desirable to be able to print any possible sequence. The method disclosed herein entails assembling nucleic acids in a way that is sequence independent, therefore is ideal for assembling nucleic acids as information.

While this patent document contains many specifics, these should not be construed as limitations on the scope of any invention or of what may be claimed, but rather as descriptions of features that may be specific to particular embodiments of particular inventions. Certain features that are described in this patent document in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. Moreover, the separation of various system components in the embodiments described in this patent document should not be understood as requiring such separation in all embodiments.

Only a few implementations and examples are described and other implementations, enhancements and variations can be made based on what is described and illustrated in this patent document.

1. A method of assembling nucleic acid fragments, comprising:
(a) attaching a first nucleic acid fragment to a solid phase; and
(b) contacting the first nucleic acid fragment with a second nucleic acid fragment such that the second nucleic acid fragment is ligated to the first nucleic acid fragment.

2. The method of claim 1, further comprising:
(c) recovering the ligated nucleic acid to obtain the assembled nucleic acid.

3. The method of claim 1 or claim 2, wherein step (b) is carried out in the presence of a ligase.

4. The method of claim 2 or claim 3, wherein the assembled nucleic acid is recovered by amplification or by cleaving the assembled nucleic acid from the solid phase.

5. The method of any one of claims 1 to 4, wherein the nucleic acid fragment to be assembled is single-stranded or double stranded.

6. The method of claim 5, wherein the method further polymerizing the single-stranded nucleic acid into double-stranded before, after, or during the ligation step.

7. The method of any one of claims 1 to 6, wherein the nucleic acid fragments are ligated by blunt ligation.

8. The method of any one of claims 1 to 6, wherein the nucleic acid fragments are ligated by end-to-end ligation.

9. The method of any one of claims 1 to 8, wherein the nucleic acid fragments to be assembled are non-overlapping.

10. The method of any one of claims 2 to 9, after step (b) or before step (c), further comprising:
removing excessive second nucleic acid fragment;
phosphorylating the ligated nucleic acid; and
contacting the ligated nucleic acid with a third nucleic acid fragment such that the third nucleic acid fragment is ligated to the second nucleic acid fragment.

11. The method of claim 9, wherein the steps are repeated one or more times to assemble one or more additional nucleic acid fragments.

12. The method of any one of claims 1 to 11, wherein the solid phase has a flat surface, a circular surface or a sphere surface.

13. The method of any one of claims 1 to 12, wherein the nucleic acid fragments to be assembled comprise DNA, RNA, locked nucleic acid (LNA), unlocked nucleic acid (UNA), or peptide nucleic acid (PNA) fragments.

14. The method of any one of claims 1 to 13, wherein the first nucleic acid fragment is directly attached to the surface of the solid phase via a covalent linkage or a non-covalent linkage.

15. The method of any one of claims 1 to 13, wherein the first nucleic acid fragment is indirectly attached to the surface of the solid phase via an anchoring oligo.

16. The method of claim 15, wherein the anchoring oligo is single-stranded or double-stranded.

17. The method of any one of claims 1 to 16, wherein the 3′ end of the first nucleic acid fragment is attached to the solid phase.

18. The method of any one of claims 1 to 16, wherein the 3′ end of the first nucleic acid fragment is blocked or capped such that one or more additional nucleic acid fragments to be assembled are extended from 5′ end only.

19. The method of any one of claims 1 to 18, wherein the second nucleic acid fragment or additional nucleic acid fragments are not immobilized on the solid phase.

20. A system for assembling nucleic acid fragments, comprising:
a solid phase;
a first nucleic acid fragment immobilized on the surface of the solid phase; and
one or more nucleic acid fragments to be assembled,
wherein 3′ end of the first nucleic acid fragment is attached to the surface of the solid phase or is blocked or capped such that the one or more nucleic acid fragments can be assembled to 5′ end only.

21. The system of claim 20, further comprising a ligase and a kinase.

22. The system of claim 20 or claim 21, further comprising a polymerase and a nuclease.

23. The system of any one of claims 20 to 22, further comprising an anchoring oligo attached to the solid phase, and the first nucleic acid fragment is linked to the anchoring oligo.
